(19) United States
(12) Patent Application Publication
(10) Pub. No.: US 2026/0007819 A1
(43) Pub. Date: Jan. 8, 2026

(54) Nutritional Infusion Device, Nutritional Infusion Tubing and Nutritional Infusion System

(71) Applicant: Medcaptain Medical Technology Co., Ltd., Shenzhen (CN)

(72) Inventors: Pan HU, Shenzhen (CN); Xiang LI, Shenzhen (CN); Zhao WU, Shenzhen (CN); Zhiwu DENG, Shenzhen (CN)

(73) Assignee: Medcaptain Medical Technology Co., Ltd., Shenzhen (CN)

(21) Appl. No.: 19/258,579

(22) Filed: Jul. 2, 2025

(30) Foreign Application Priority Data
Jul. 4, 2024 (CN) 202410893456.9
Jul. 4, 2024 (CN) 202410893470.9
Jul. 4, 2024 (CN) 202421579668.1

(51) Int. Cl. A61M 5/142; A61J 15/00

(52) U.S. Cl. CPC A61M 5/14232; A61J 15/0076

(57) ABSTRACT

A nutritional infusion system includes an infusion device and a nutritional infusion tubing assembly. The nutritional infusion tubing assembly includes infusion tubing, which in turn includes a peristaltic tube. The nutritional infusion device includes a housing, a power supply, a control assembly, a driving component, a roller, and a first fixing member. The housing has a mounting panel, and the mounting panel has a guide rail. The guide rail guides movement and placement of the nutritional infusion tubing assembly, which is provided with a guide channel. The cooperation between the guide channel and the guide rail reduces interference from elastic resistance generated by stretching the peristaltic tube during installation. This allows the nutritional infusion tubing to be smoothly installed, even with only one hand, on the nutritional infusion device by applying only a force along the extension direction of the guide rail.

### REFERENCE TO RELATED APPLICATIONS

The invention claims priority of Chinese Patent Application No. 202410893456.9, entitled “Method of Assembling Nutritional infusion tubing for Nutritional Infusion Device and Nutritional infusion system” filed with the China National Intellectual Property Administration on Jul. 4, 2024; Chinese Patent Application No. 202410893470.9, entitled “Nutritional Infusion Device, Nutritional infusion tubing and Nutritional infusion system” filed with the China National Intellectual Property Administration on Jul. 4, 2024; and Chinese Patent Application No. 202421579668.1, entitled “Nutritional Infusion Device, Nutritional infusion tubing and Nutritional infusion system” filed with the China National Intellectual Property Administration on Jul. 4, 2024, which are incorporated herein by reference in its entirety.

### TECHNICAL FIELD

This invention generally relates to the technical field of medical devices and more particularly to a nutritional infusion device, nutritional infusion tubing, and a nutritional infusion system.

### BACKGROUND ART

In the medical device field, enteral feeding pumps are used to provide liquid nutrition to patients unable to eat. An enteral feeding pump system typically includes a nutrition infusion device and disposable nutritional infusion tubing, designed to pump either liquid nutritional formula alone or both formula and water. During installation, the nutrition delivery tubing needs to be stretched to a certain degree before being secured. Due to the tubing's inherent resilience, the stretching process requires that the tubing's elastic resistance to be overcome, making installation physically strenuous. Moreover, successful installation demands coordinated use of both hands by the operator, resulting in cumbersome operation and an overly complicated assembly procedure.

### SUMMARY OF THE INVENTION

This invention proposes a nutritional infusion device and a nutritional infusion tubing assembly, which together form a nutritional infusion system. In this invention, by providing a guide channel in the nutritional infusion device and a guide rail in the nutritional infusion tubing assembly, the interference during assembly of the system caused by the elastic resistance due to stretching of the nutritional infusion tubing is reduced by the cooperating effect of the guide channel and the guide rail, which facilitates smooth mounting of the nutritional infusion tubing on the nutritional infusion device.

In a first aspect, embodiments of this invention provide a nutritional infusion device, comprising:
a housing;
a power supply and a control assembly provided inside the housing;
a driving assembly connected to the control assembly and provided inside the housing;
a roller connected to the driving assembly and exposed above a mounting panel of the housing; wherein the roller is configured for a peristaltic tube of the nutritional infusion tubing to be looped around it; under the drive of the driving assembly, the roller is configured to drive a plurality of rotors set in the roller to sequentially squeeze the peristaltic tube so as to cause a directional movement of a liquid inside the peristaltic tube; and
a first fixing member;
wherein the mounting panel is provided with a guide rail, which is configured to provide a guiding effect for the movement of the nutritional infusion tubing with respect to the mounting panel after the nutritional infusion tubing has been looped around the roller and before it is fixed to the first fixing member.

In a second aspect, embodiments of this invention provides nutritional infusion tubing, comprising:
a peristaltic tube;
infusion tubing comprising an input tube and an output tube;
an input tube connector connected to the input tube and one end of the peristaltic tube;
an output tube connector connected to the output tube and the other end of the peristaltic tube;
a support plate provided with a first mounting groove and a second mounting groove, wherein the first mounting groove is configured to accommodate the input tube connector, and the second mounting groove is configured to accommodate the output tube connector; and
a second mounting member fixedly connected to a side of the support plate back away from the peristaltic tube.

In the second aspect, embodiments of this invention provide a nutritional infusion device as described in the first aspect and nutritional infusion tubing as described in the second aspect, wherein the mounting panel faces the support plate, the peristaltic tube is looped on the rotor of the roller, the guide rail abuts an inner wall of the guide channel, and the first fixing member is detachably connected to the second fixing member.

### BRIEF DESCRIPTION OF THE DRAWINGS

FIG. 1 is a schematic structural diagram of a nutritional infusion system according to some embodiments of this invention.

FIG. 2 is a schematic structural diagram of the nutritional infusion system shown in FIG. 1 in another state.

FIG. 3 is a schematic structural diagram of nutritional infusion tubing shown in FIG. 2 according to some embodiments of this invention.

FIG. 4 is a schematic structural diagram of the nutritional infusion tubing shown in FIG. 3 in another view.

FIG. 5 is a partial structural diagram of the nutritional infusion device shown in FIG. 2 according to some embodiments of this invention.

FIG. 6 is a schematic structural diagram of a pump door shown in FIG. 2 in another view.

FIG. 7 is a schematic structural diagram of a nutritional infusion device according to some embodiments of this invention.

FIG. 8 is a partially enlarged structural schematic diagram of area A in FIG. 7.

FIG. 9 is a schematic diagram of a mating structure between the guide rail and the mounting panel according to some embodiments of the present application.

FIG. 10 is a schematic structural diagram of the nutritional infusion tubing according to some embodiments of this invention.

FIG. 11 is a partially enlarged structural schematic diagram of area B in FIG. 10.

FIG. 12 is an exploded view of the nutritional infusion tubing of FIG. 10.

FIG. 13 is another exploded view of the nutritional infusion tubing of FIG. 10.

FIG. 14 is a schematic structural diagram of the nutritional infusion system according to some embodiments of this invention.

FIG. 15 is a schematic diagram of the mating structure of the roller and the peristaltic tube according to some embodiments of this invention.

FIG. 16 is a schematic diagram of the mating structure of the guide rail and a guide channel according to some embodiments of this invention.

FIG. 17 is a schematic diagram of a working system of a roller according to an embodiment of this invention.

FIG. 18 is a flowchart of a method of assembling the nutritional infusion tubing assembly and the nutritional infusion device according to an embodiment of this invention.

FIG. 19 is a schematic diagram of an assembly process of the nutritional infusion system made from the method shown in FIG. 18.

FIG. 20 is a schematic structural diagram of another nutritional infusion tubing assembly according to some embodiments of the present application.

### DETAILED DESCRIPTION

The embodiments of this invention are described below in connection with the accompanying drawings of the embodiments of this invention.

In the description of the embodiments of this invention, it is to be noted that, unless otherwise expressly provided and limited, the terms “mount” and “connect” are to be broadly construed. For example, “connected” may be detachably connected or non-detachably connected, directly connected or indirectly connected through an intermediate medium. “Plurality” means at least two.

Orientation terms referred to in embodiments of this invention, such as “up”, “inner”, “outer”, “top” “bottom”, “side” are only orientations with reference to the accompanying drawings. Accordingly, the orientation terms used are intended to better and more clearly illustrate and understand the embodiments of the present application, and are not intended to indicate or imply that the device or element referred to must have a particular orientation, be constructed and operated in a particular orientation, and therefore are not to be construed as a limitation of the embodiments of this invention.

In the embodiments of this invention, the specified relative positional relationships (e.g., parallel, perpendicular) refer to the current technological level and are not absolute, strict limitations. Minor deviations are permitted, including being approximately parallel or approximately perpendicular. For example, ‘A is parallel to B’ means that A and B are either perfectly parallel or approximately parallel, with an included angle between 0 and 10 degrees. Similarly, ‘A is perpendicular to B’ indicates that A and B are either precisely perpendicular or approximately perpendicular, with an included angle ranging from 80 to 100 degrees

In the embodiments of this invention, the terms “first” and “second” are used for descriptive purposes only and are not to be understood as indicating or implying relative importance or implicitly specifying the number of technical features indicated. As a result, the feature defined with “first” or “second” may expressly or implicitly include one or more such features. In this regard, the one-piece structural member means that one of the parts of the structural member is connected to the other part during the process of forming the other part, and there is no need to connect the two parts together by means of further processing (e.g., adhesive bonding, welding, snap tab connection, screw connection).

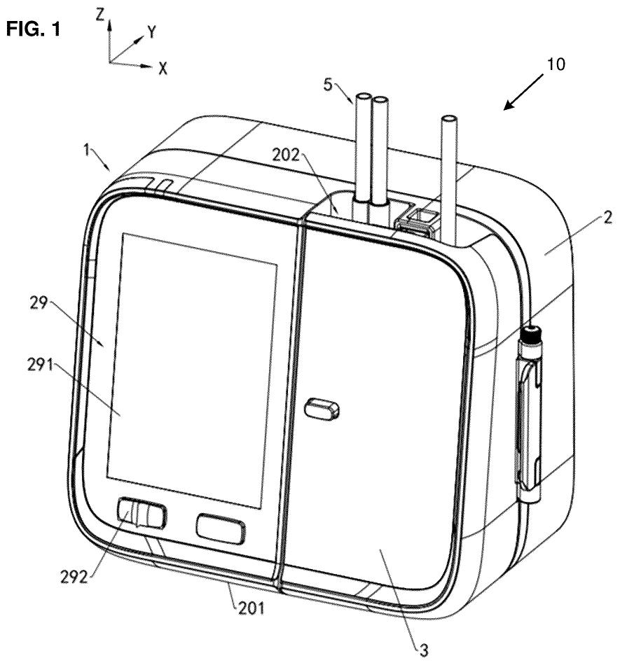

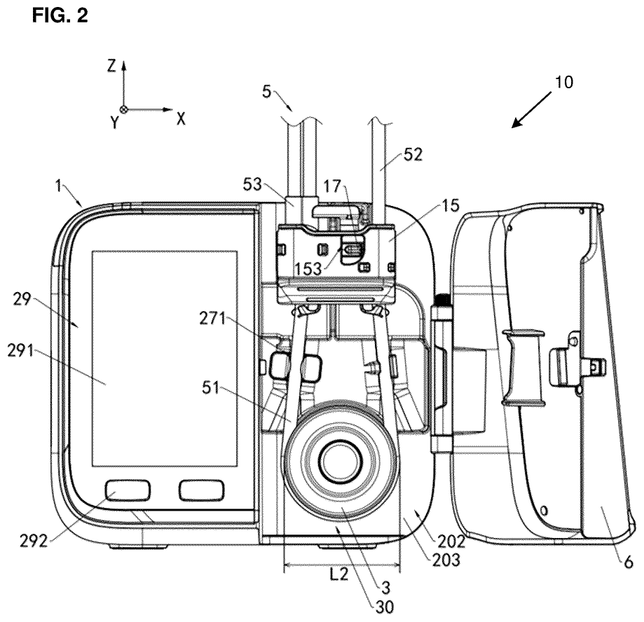

As shown in FIG. 1 and FIG. 2, in some embodiments, the nutritional infusion system 10 may include a nutritional infusion tubing assembly 5 and a nutritional infusion device 1, which, together form the nutritional infusion system. In other words, when the nutritional infusion tubing assembly is mounted in the nutritional infusion device, together they form the nutritional infusion system 10. The nutritional infusion tubing assembly 5 may be detachably mounted on the nutritional infusion device 1. In clinical practice, the nutritional infusion system 10 is generally utilized to deliver water, nutrient solution, and pre-prepared food emulsion of a certain concentration to the patient, thereby providing nutritional support. The nutritional infusion device 1 may provide a driving force for the fluid within the nutritional infusion tubing assembly 5 mounted thereto to push and deliver the fluid within the nutritional infusion tubing assembly 5 to the patient.

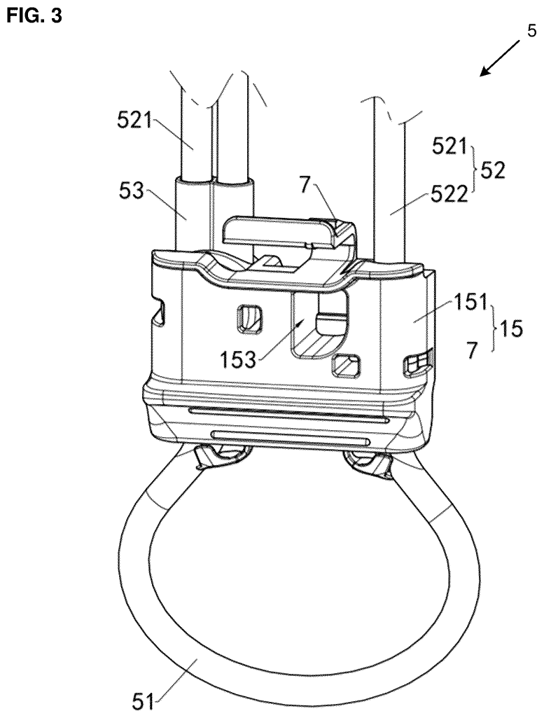

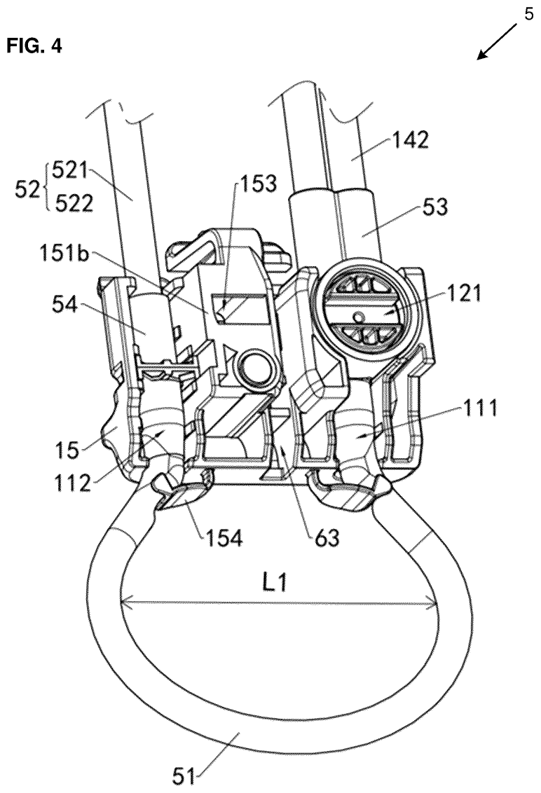

As shown in FIG. 3 and FIG. 4, in some embodiments, the nutritional infusion tubing assembly 5 may include a peristaltic tube 51, an input tube connector 53, an output tube connector 54, infusion tubes 52, and a support plate 15. The input tube connector 53 and the output tube connector 54 are both fixed to the support plate 15. One end of the input tube connector 53 is connected to an input end 111 of the peristaltic tube 51, and the opposite end thereof is connected to one of the infusion tubes 52. One end of the output tube connector 54 is connected to an output end 112 of the peristaltic tube 51, and its opposite end is connected to another infusion tubes 52. By connecting the peristaltic tube 51, the input tube connector 53, the output tube connector 54, and the infusion tubes 52, it is possible to form a one-piece structure by assembling the tubing portion within the nutritional infusion tubing assembly 5, which improves the degree of modularity of the structure within the nutritional infusion tubing assembly 5, and is conducive to reducing the difficulty of moving and installing the nutritional infusion tubing assembly 5. In addition, by providing the support plate 15 to connect the input tube connector 53 and the output tube connector 54, the support plate 15 can be used to fix the relative positions of the input tube connector 53 and the output tube connector 54, which is conducive to maintaining the structural shape of the nutritional infusion tubing assembly 5, thereby further improving the integration of its and reducing the operational difficulty of moving the nutritional infusion tubing assembly 5.

As an example, the peristaltic tube 51 may be made of materials such as silicone, which has high electrical heat resistance, insulation, reliability, bendability, and flexibility. In embodiments of this invention, the peristaltic tube 51 may be directly looped around the input tube connector 53 and/or the output tube connector 54 using its elasticity, or it may be connected to the input tube connector 53 and/or the output tube connector 54 by means of adhesive bonding, ultrasonic welding, etc. In other embodiments, the peristaltic tube 51 and the input tube connector 53 and/or the output tube connector 54 may be integrated into a single structure, which is not limited in this invention. In some embodiments, the input tube connector 53 and/or the output tube connector 54 may not be included in the nutritional infusion tubing assembly 5, one end of the peristaltic tube 51 may be directly connected to one infusion tubes 52, and the other end of the peristaltic tube 51 may be directly connected to another infusion tube 52; the invention is not limited in this regard.

As an example, the number of infusion tubes 52 may be two, three or more and serve as a fluid delivery path. The plurality of infusion tubes 52 may include an output tube 522 and at least one input tube 521, and the plurality of input tubes 521 may be used for supplying different fluids such as nutrient fluid, cleaning fluid, etc. into the peristaltic tube 51, respectively, which are then transported to the patient through the output tube 522. The infusion tubes 52 may be made of synthetic materials such as polyvinyl chloride (PVC) with additives or thermoplastic polyurethane elastomer rubber (TPU)—this invention is not limited to these material choices.

In some examples, the input tube connector 53 may be a valve mechanism such as a two-way valve with one inlet and one outlet or a three-way valve with two inlets and one outlet, whereby the position of the valve core in the control valve is controlled to control the connectivity of the passage between the infusion tubes 52 and the peristaltic tube 51. For example, the infusion tube(s) 52 may be connected to one of the inlets and the outlet of the peristaltic tube 51, for another example, the infusion tubes 52 may be connected to the other inlet and the outlet of the peristaltic tube 51. As an alternative, the passage between the infusion tubes 52 and the peristaltic tube 51 may be blocked, so as to realize on-off and switching of the fluid pathway in the nutritional infusion tubing assembly 5. The valve mechanism is more flexible and reliable, which helps to improve the reliability of the nutritional infusion tubing assembly 5 and the nutritional infusion system 1. Before the nutritional infusion tubing assembly 5 is mounted on the nutritional infusion device 1, it may be in a blocked state. After the nutritional infusion tubing assembly 5 is mounted on the nutritional infusion device 1, the three-way/two-way valves are controlled by rotating the angle of the valve core to connect to the corresponding tube according to the setting to realize the desired nutritional fluid feeding. After stopping feeding, the three-way/two-way valve core is reset to a blocking state by rotating the control valve core to avoid uncontrolled free flow of the nutritional infusion tubing assembly 5 after it is removed from the nutritional infusion device 1, so as to improve the reliability and safety of the nutritional infusion system 1. In other embodiments, the input tube connector 53 may also have other structures; the invention is not limited in this regard.

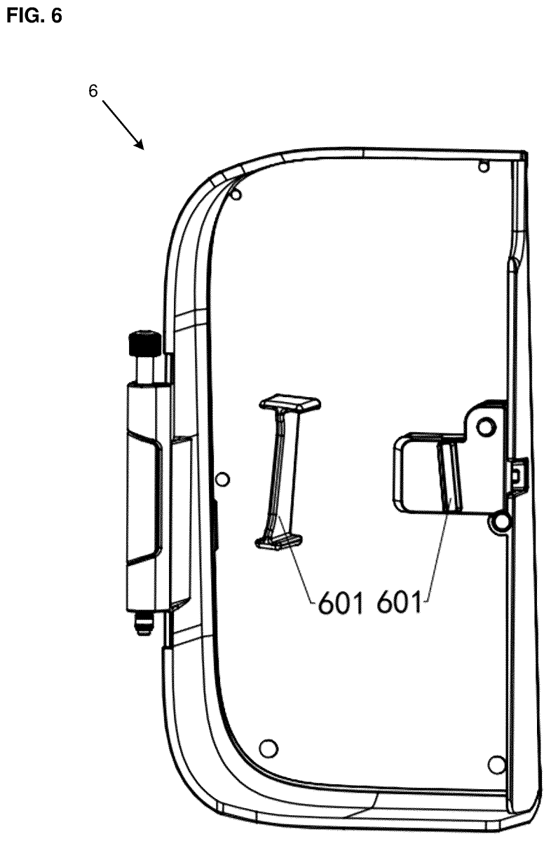

As shown in FIGS. 1-2 and 5-6, in some embodiments, the nutritional infusion device 1 may include a housing 21 and a pump door 6, which may be rotatably connected to a side of the housing 21. As an example, the housing 21 may be made of a material such as plastic, silicone, or the like, for providing supporting strength for the nutritional infusion device 1. The pump door 6 may be hinged to the housing 21 via a door pivot. In some other embodiments, the pump door 6 may also be detachably connected to the pump body and closed on the housing 21 when needed, or the pump door 6 may be slidably connected to the housing 21. The invention is not limited to any particular manner in which the pump door 6 is connected to the housing 21. In some embodiments, the nutritional infusion device 1 may also omit the pump door 6; the invention is not limited to this option either.

As an example, the nutritional infusion device 1 may be provided with a mounting space 202, which may be considered to be formed by a portion of the outer surface of the housing 21 being recessed towards the interior of the housing 21, and may be used to organize other structural members (e.g., nutritional infusion tubing assembly 5) within the nutritional infusion system 10. During the opening of the pump door 6 with respect to the housing 21, the structural members mounted within the mounting space 202 are exposed to facilitate an operator to install or replace the structural members in the mounting space 202. When the pump door 6 is closed relative to the housing 21, the pump door 6 can cover the mounting space 202 for protecting the structural members mounted in the mounting space 202, avoiding external impurities (e.g., water, dust) from affecting the service life of the structural members in the mounting space 202. As shown in FIG. 1, the pump door 6 and the housing 21 may together form the outer contour of the nutritional infusion system 10. When the pump door 6 is closed relative to the housing 21, the overall shape of the nutritional infusion system 10 can be restored, and the outer surface of the nutritional infusion system 10 may be considered approximately planar, which ensures the aesthetics of the product.

As an example, the nutritional infusion device 1 may be placed on a support base such as a tabletop or support frame. The nutritional infusion device 1 may have a bottom surface 201, which may be configured to contact a support base such as a tabletop or a support frame. In some examples, the bottom surface 201 of the nutritional infusion device 1 may also be provided with a flexible support pad to avoid hard contact between the nutritional infusion device 1 and the support base, to increase the friction between the nutritional infusion device 1 and the support base, to ensure the stability of the nutritional infusion device 1, and also to slow down the abrasion or scratching of the bottom surface 201 of the nutritional infusion device 1. The side on which the bottom surface 201 of the nutritional infusion device 1 is located is defined as a bottom side of the nutritional infusion device 1, and the top side of the nutritional infusion device 1 is opposite the bottom side.

It should be understood that, for clarity of description, in the embodiments of this invention, the nutritional infusion device 1 shown in FIG. 1 is defined to be in an upright state, and the nutritional infusion device 1 shown in FIG. 1 is defined to have an X-axis direction, a Y-axis direction, and a Z-axis direction. The X-axis direction may be parallel to a length direction of the nutritional infusion device 1, the Z-axis direction may be parallel to a height direction of the nutritional infusion device 1, and the Y-axis direction may be parallel to a width direction of the nutritional infusion device 1. Any two of the X-axis direction, the Y-axis direction and the Z-axis direction are perpendicular to each other. In other embodiments, the nutritional infusion device 1 may be positioned horizontally, suspended, or mounted in other orientations on supporting bases such as tabletops or brackets. The coordinate system configuration may be flexibly adapted to practical requirements, with no limitation imposed by the present application.

In some embodiments, the nutritional infusion device 1 may also include a driving assembly 30 and a roller 3. The roller 3 may be mounted in the mounting space 202 and rotatably connected to the housing 21. As an example, the housing 21 may have a mounting surface 203. An operator may then face the mounting surface 203 of the nutritional infusion device 1 and operate the structural members located in the mounting space 202, and the roller 3 may be exposed relative to the mounting surface 203 of the housing 21. In an embodiment of this invention, the roller 3 may be provided close to a bottom side of the nutritional infusion device 1. In other embodiments, the roller 3 may be provided close to a top side of the nutritional infusion device 1, or the roller 3 may be provided in an intermediate position of the nutritional infusion device 1, none of which is limited by this invention.

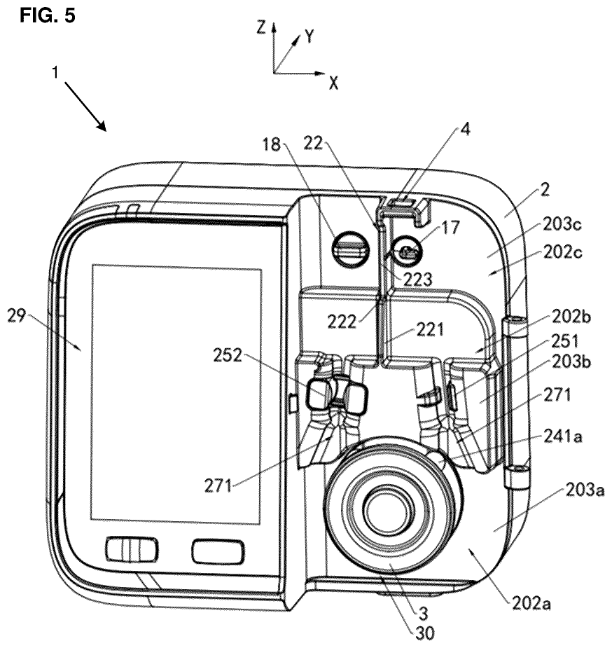

As shown in FIG. 2, in an embodiment of this invention, the tubing of the nutritional infusion tubing assembly 5 may be looped in a groove around an outer periphery of the roller 3 such that the roller 3 is capable of exerting a certain pressure on the tubing. For example, the peristaltic tube 51 may abut at least a portion of the roller 3be stretched when the roller 3 is rotated. As an example, as shown in FIG. 5, a plurality of rotors 241a may be provided on a circumferential side of the roller 3. For example, there may be four rotors 241a that may spaced apart along the circumference of the roller 3. The peristaltic tube 51 may at least partially abut the rotors 241a on the roller 3. The peristaltic tube 51 between the two rotors 241a may then exhibit a pillow-shaped deformation when stretched. As the roller 3 rotates, it alternately squeezes and releases the peristaltic tube 51 to propel the fluid.

In some embodiments, the driving assembly 30 may also include a first motor (not shown in the figures), which may be drivingly connected to the roller 3 and mounted in an inner cavity (not shown in the figures) of the housing 21. When the first motor operates to drive the roller 3 to rotate, the roller 3 is able to generate a pushing force on the fluid within the pipeline of the peristaltic tube 51, pushing the fluid within the pipeline of the peristaltic tube 51 to flow along the pipeline to deliver the nutrient flowing into the peristaltic tube 51 to the patient; when the roller 3 stops rotating, the pipeline of the peristaltic tube 51 is subjected to a higher pressure from the roller 3, and the peristaltic tube 51's pipeline fluid pathway is equivalent to being blocked, the fluid in the pipeline of the peristaltic tube 51 stops continuing to flow forward, and then the delivery of the nutrient to the patient is stopped. In this embodiment of the invention, the first motor is used as the driving source to drive the roller 3 to rotate. In other embodiments, other driving sources may be used to drive the roller 3 to move.

In some embodiments, the nutritional infusion device 1 may also include a locking member 17, the support plate 15 may be provided with a locking hole 153, and the locking member 17 may be slotted in the locking hole 153 to realize a limiting effect on the nutritional infusion tubing assembly 5. The locking member 17 may be slidably connected or rotatably connected to the mounting surface 203 of the housing 21, and the limiting effect of the support plate 15 in other directions may be realized by rotating or moving the locking member 17. It should be understood that, for example, in the embodiment of this invention, as shown in FIG. 2, the locking hole 153 is provided with a first stopping piece and a second stopping piece spaced apart along the Z-axis direction. In the initial position, the locking member 17 may extend along the X-axis direction, so the locking member 17 may be slotted in the space between the first stopping piece and the second stopping piece to block the movement of the support plate 15 with respect to the housing 2 in the Z-axis direction. By rotating the locking member 17, the position of the locking member 17 may be changed, and it may also be possible to make the locking member 17 extend along the Z-axis to be abutted against the space between the first stopping piece and the second stopping piece to realize the obstruction of the movement of the support plate 15 in the Y-axis direction relative to the housing 2. The embodiments of this invention are not limited to any particular connection method between the locking stop member 17 and the housing 2, the relative position of the locking stop member 17 and the locking hole 153, and the like. In some other embodiments, the locking member 17 and the locking holes 153 may not be included within the nutritional infusion system 10 at all.

In some embodiments, the driving assembly 30 may also include a second motor (not shown in the drawings), which may be drivingly connected to the locking member 17 and be mounted within the inner cavity of the housing 21. By controlling the operation of the second motor to drive the locking member 17 to move, the degree of automation of the nutritional infusion system 1 can be improved; this helps lower the difficulty the operator may experience using the nutritional infusion system 1. In the embodiments of this invention, the second motor is used as a driving source to drive the locking member 17 to move. In other embodiments, other driving sources may be used to drive the locking member 17 to move.

In some embodiments, the nutritional infusion device 1 may also be provided with a switching member 18; the valve structure of the nutritional infusion tubing assembly 5 may be provided with a reversing groove 121; and the switching member 18 may be slotted in the reversing groove 121 of the valve structure to drive the valve spool of the valve structure to rotate, so as to control the switching of the valve spool between different positions, so as to realize the on/off and the switching of the fluid pathway within the nutritional infusion tubing assembly 5.

In some embodiments, the driving assembly 30 may also include a third motor (not shown in the drawings), which may be drivingly connected to the switching member 18, and be mounted within the inner cavity of the housing 21. By controlling the work of the third motor to drive the movement of the switching member 18, the degree of automation of the nutritional infusion system 10 can be improved, which is conducive to lowering the operating difficulty of the operator in using the nutritional infusion system 10. In some embodiments, a transmission mechanism such as a gear may also be provided between the switching member 18 and the locking member 17 to realize driving the second motor or the third motor while driving the switching member 18 and the locking member 17 together, which helps reduce the number of structural members within the nutritional infusion device 1. Therefore, the nutritional infusion device 1 may include the second motor without including the third motor or it may include the third motor without including the second motor, which helps reduce the manufacturing cost of the nutritional infusion system 10.

As shown in FIG. 2 and FIG. 5, structures such as tabs or grooves may also be provided on the mounting surface 203, to divide the mounting space 202 into several interconnected, but regions of different depth to better arrange and mount structural members of different sizes within the mounting space 202. In some examples, the mounting space 202 may include a first mounting space 202a, a second mounting space 202b, and a third mounting space 202c sequentially connected in the Z-axis direction. In the Y-axis direction, the the first mounting space 202a is located deeper that the second mounting space 202b, and the third mounting space 202c is located at a depth than the second mounting space 202b. As an example, in the embodiments of this invention, the mounting surface 203 may also be considered to be divided into a first mounting surface 203a, a second mounting surface 203b, and a third mounting surface 203c. The first mounting surface 203a lies within the first mounting space 202a; the second mounting surface 203b lies within the second mounting space 203b, and the third mounting surface 203c lies within the third mounting space 203c. The first mounting surface 203a, the second mounting surface 203b, and the third mounting surface 203c are oriented in the same direction. That is, in the embodiments of this invention, the roller 3 may be disposed on the first mounting surface 203a of the first mounting space 202a, the tube groove 271 may be disposed on the second mounting surface 203b of the second mounting space 202b, and the first fixing member 4, the locking member 17, and the switching member 18 may be disposed on the third mounting surface 203c of the third mounting space 202c. As shown in FIG. 2, when the nutritional infusion tubing assembly 5 is mounted in place, the support plate 15 may be disposed within the third mounting space 202c, a portion of the peristaltic tube 51 is slotted in the tube groove 271 within the second mounting space 202b, and the peristaltic tube 51 may tightly abut at least a portion of the roller 3. The first mounting space 202a, the second mounting space 202b, and the third mounting space 202c may also, as one option, be divided into a plurality of sub-mounting spaces with different longitudinal depths. In other embodiments, the dimensional relationship of the first mounting space 202a, the second mounting space 202b, and the third mounting space 202c may not be the same as in the embodiments of this invention, and the mounting space 202 may be divided into more or fewer zones.

In some embodiments, the nutritional infusion device 1 may also have a display control area 29, which is mainly configured for displaying information related to the nutritional infusion device 1, so that an operator may operate in the display control area 29 to control the operating state of the nutritional infusion device 1.

As an example, the display control area 29 may be provided with a display screen 291 and buttons 292. The display screen 291 may occupy a large portion of the display control area 29, and the buttons 292 may be provided on the peripheral side of the display screen 291. The display screen 291 may be configured to display information such as operating parameters and functional options of the nutritional infusion device 1, and the buttons 292 may include an on/off key, a return key, etc. The display control area 29 is preferably positioned so that it is visible even when the pump door 6 is closed so that an operator can observe the display screen 291 and manipulate the nutritional infusion device 1. The buttons 292 may be used to manipulate the working state of the different members within the nutritional infusion device 1 (e.g., to manipulate elements such as a first motor connected to the roller 3 and a second motor connected to the locking member 17), so as to increase the degree of automation of the nutritional infusion system 10, as well as to reduce the risk of the nutritional infusion system 10 being used incorrectly or without authorization. In some embodiments, the display screen 291 may also be a touch screen, and the buttons 292 may not be provided in the display control area 29.

It should be understood that the shapes, sizes, etc. of the nutritional infusion system 10, the nutritional infusion device 1, and the nutritional infusion tubing assembly 5 shown in the FIGS. 1 to 5 are only schematic representations. In other embodiments, they may be adjusted as needed.

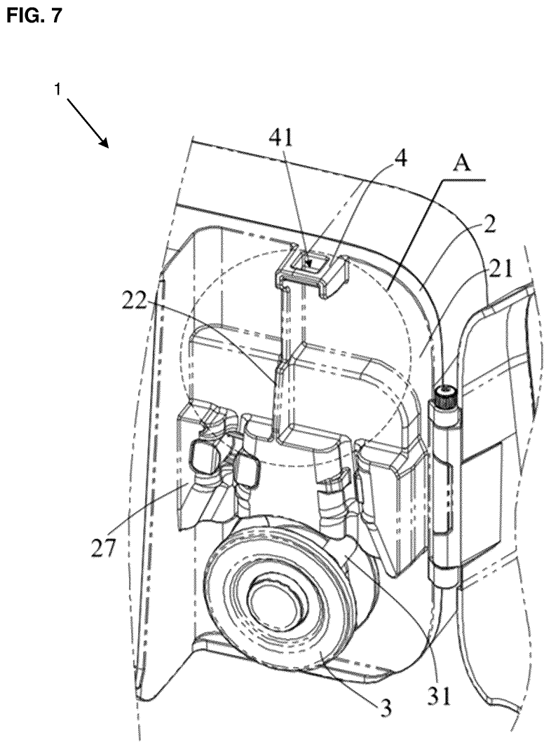

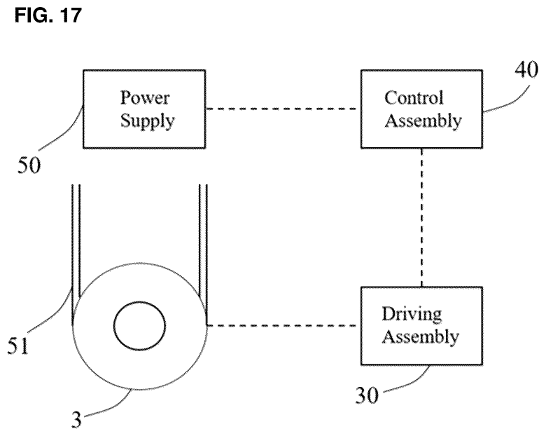

FIGS. 7, 10, and 14 are schematic structural diagrams of various embodiments of a nutritional infusion device according to some embodiments of this invention, and FIG. 17 is a schematic diagram of a working system of a roller according to an embodiment of this invention. As shown in FIGS. 7, 10, 14 and 17, the nutritional infusion device 1 may be used with the nutritional infusion tubing assembly 5, and may include a housing 20, a power supply 50, a control assembly 40, a driving assembly 30, a roller 3 and a first fixing member 4. The driving assembly 30 is connected to the control assembly 40, and the driving assembly 30, the assembly 40, and the power supply 50 may be all disposed inside the housing 2. The roller 3 may be connected to the driving assembly and exposed on the mounting panel 21 of the housing 2. The roller 3 may be configured for the peristaltic tube 51 of the nutritional infusion tubing assembly 5 to be looped around it. Under the drive of the driving assembly, the roller 3 is configured to cause a plurality of rotors 31 set in the roller 3 to sequentially squeeze the peristaltic tube 51, so as to cause a directional movement of the liquid inside the peristaltic tube 51. The mounting panel 21 may be provided with a guide rail 22, which is configured to guide the movement of the nutritional infusion tubing assembly 5 with respect to the mounting panel 21 after the nutritional infusion tubing assembly 5 has been looped/looped around the roller 3 and before it has been fixed to the first fixing member 4.

By providing a guide rail 22 on the mounting panel 21 of the housing 2, after the peristaltic tube 51 of the nutritional infusion tubing assembly 5 has been looped around the roller 3, the influence of the rebound force on the nutritional infusion tubing assembly 5 can be limited by the action of the guide rail 22 along the extension direction of the guide rail 22, i.e. the component of the rebound force along the other directions can be canceled out by the guide rail 22, thus making it possible for the nutritional infusion tubing assembly 5 to be subjected to an extension direction force only along the guide rail 22. The nutritional infusion device 1 can be smoothly mounted on the nutritional infusion device 1 by applying the force in the extension direction of the guide rail 22.

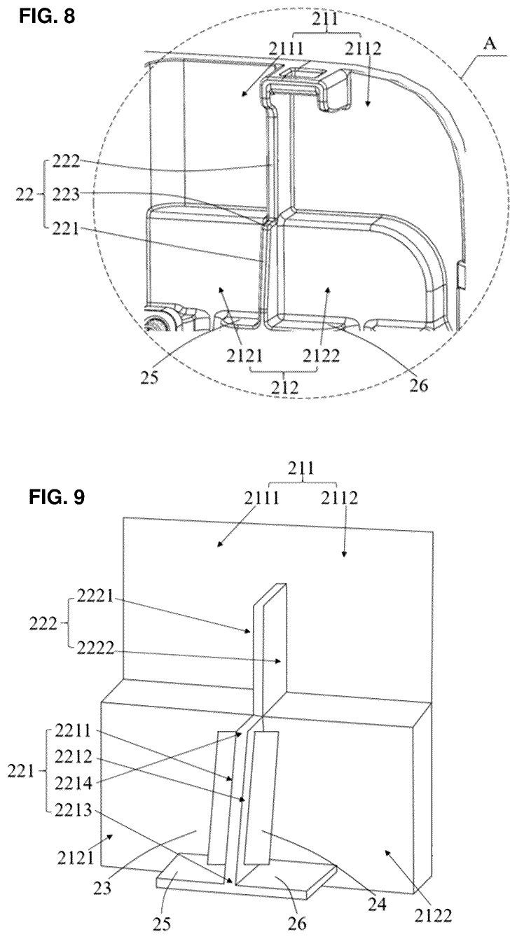

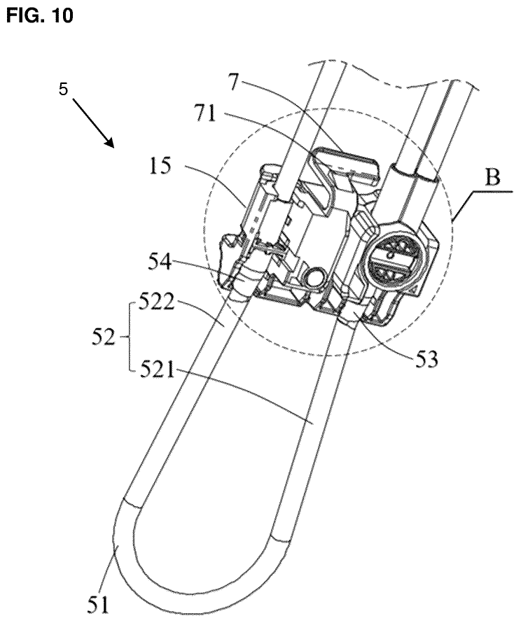

FIG. 8 is a partially enlarged structural schematic diagram of area A in FIG. 7. As shown in FIG. 7, FIG. 8 and FIG. 10, in a possible embodiment, the mounting panel 21 is provided with a recess 211, at least a partial wall of the recess 211 may be configured to abut against at least a partial portion of the support plate 15 of the nutritional infusion tubing assembly 5, after the support plate 15 of the nutritional infusion tubing assembly 5 has been fixed to the first fixing member 4. As an example, the guide channel 63 provided on the support plate 15 may be abutted against the at least partial wall of the recess 211, or parts of the support plate 15 other than the guide channel 63 may be abutted against the at least partial wall of the recess 211. By providing the recess 211 on the mounting panel 21, after the nutritional infusion tubing assembly 5 has been mounted on the nutritional infusion device 1, at least a partial portion of the recess 211 may provide a supporting force on the support plate 15 of the nutritional infusion tubing assembly 5. As an example, the direction of the supporting force may be directed from the roller 3 to the first fixing member 4, while a snap groove 41 in the first fixing member 4 may exert an abutting force on a corresponding structure of the nutritional infusion tubing assembly 5, such as a snap tab 71. The direction of the resisting force may be parallel with or intersect the plane where the mounting panel 21 is located. With the combined effect of the supporting force and the resisting force, i.e. the recess 211 and the first fixing member 4, the nutritional infusion tubing assembly 5 may be prevented from falling off from the nutritional infusion device 1 during operation.

As shown in FIGS. 7, 8, and 10, in one possible embodiment, the first fixing member 4 may include the snap groove 41, and the second fixing member 7 may include a snap tab 71. During movement of the support plate 15 along the guide rail 22, while the support plate 15 generates displacement in the direction toward the interior of the housing 2, the top of the snap tab 71 may be deformed by extrusion of the snap groove 41, and rebound at the opening of the up-to-side of the snap groove 41 which allows the snap groove 41 and the snap tab 71 to be fixed to each other. As an example, when the guide channel 63 moves along the guide rail 22 provided on the area of the mounting panel 21 without the recess 211, the snap tab 71 and snap groove 41 may be misaligned along the direction from the roller 3 toward the first fixing member 4. Conversely, when the guide channel 63 moves along the guide rail 22 provided on the recess 211, the snap tab 71 and the snap groove 41 can be aligned along the direction from the roller 3 toward the first fixing member 4. Upon abutment between at least a portion of the support plate 15 and the recess wall 211, the snap tab 71 of the nutritional infusion tubing assembly 5 engages with the snap groove 41. The synergistic effect of the supporting force provided by the recess 211 and the abutment force generated between the snap tab 71 and snap groove 41 enhances the structural stability of the mating interface between the nutritional infusion tubing assembly 5 and the nutritional infusion device 1.

As shown in FIGS. 7, 8, and 10, in one possible embodiment, the guide rail 22 may include a first guide rail 221 and a second guide rail 222, and the first guide rail 221 may be set closer to the roller 3 relative to the second guide rail 222, with the direction in which the roller 3 is facing toward the first fixing member 4 as a first direction. Both the first guide rail 221 and the second guide rail 222 may be extended in a parallel and are staggered along the first direction. As an example, a columnar connecting member may be provided between the first guide rail 221 and the second guide rail 222, and the direction between one end of the connecting member connecting the first guide rail 221 and the other end connecting the second guide rail 222 may be directed towards the recess 211, and the connecting member is configured to mutually offset the first guide rail 221 and the second guide rail 222, both extending along the first direction. Alternatively, the first guide rail 221 and second guide rail 222 may be provided continuously along the first direction, i.e., the first guide rail 221 and the second guide rail 222 may be connected end-to-end. The first guide rail 221 and the second guide rail 222 may have different slopes, and from an end of the second guide rail 222 connecting the first guide rail 221 to an end of the second guide rail 222 away from the first guide rail 221, the second guide rail 222 may be oriented towards the recess 211. The above structure facilitates that the nutritional infusion tubing assembly 5 can smoothly move along the first guide rail 221 and transition to the second guide rail 222 after being set to the roller 3, and that the nutritional infusion tubing assembly 5 can be supported by at least a partial wall of the recess 211 after it can move along the second guide rail 222 and be fixedly mounted to the first fixing member 4, in order to prevent that at least a partial wall of the recess 211, to which the nutritional infusion tubing assembly 5 is fixed to the nutritional infusion device 1, from falling off when the nutritional infusion tubing assembly 5 is working.

FIG. 9 is a schematic diagram of a mating structure between the guide rail and the mounting panel according to some embodiments of the present application. As shown in FIG. 8 and FIG. 9, in one possible embodiment, an end of the first guide rail 221 away from the second guide rail 222 may be a first end 2213, and an end of the first rail 221 connected to the second guide rail 222 may be a second end 2214, along the direction of the first end 2213 to the second end 2214. Along the direction perpendicular to the mounting panel 21, the length of the first guide rail 221 may tend to decrease from the first end 2213 to the second end 2214. As an example, the first guide rail 221 may be in the shape of a half-ladder. This may allow the height of the first guide rail 221 to gradually decrease along the direction towards the first fixing member 4, facilitating the transition of the guide channel from the first guide rail 221 to the second guide rail 222 while installing the nutritional infusion tubing assembly.

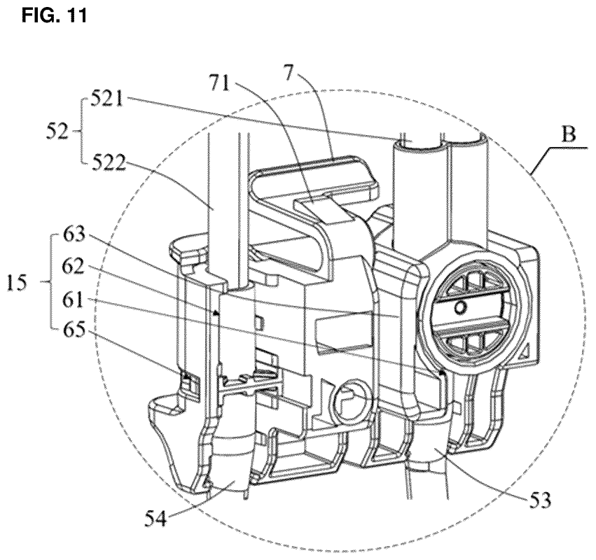

FIG. 11 is a partially enlarged structural schematic diagram of area B in FIG. 10. As shown in FIGS. 7, 8, 10, and 11, in one possible embodiment, the first guide rail 221 and the second guide rail 222 may be provided in the mounting panel 21 in a stepped shape. When the mounting panel 21 is provided with the recess 211, to ensure that the guide channel 63 can obtain support both on the non-recessed portion of the mounting panel 21 and on the inner wall of the recess 211 during its movement along the guide rail 22, the corresponding depth of the guide channel 63 needs to match the maximum height of the guide rail 22, defined as the distance between the outermost side of the second guide rail 222 and the bottom wall of the recess 211. However, by configuring the first guide rail 221 and the second guide rail 222 in a stepped arrangement on the mounting panel 21, it is possible to reduce the maximum height of the guide rail 22 while maintaining support from at least a partial wall of the recess 211 for the nutritional infusion tubing assembly 5. Consequently, the corresponding depth of the guide channel 63 in the nutritional infusion tubing assembly 5 can be reduced, resulting in a more compact mating structure between the nutritional infusion tubing assembly 5 and the nutritional infusion device 1.

As shown in FIGS. 9, 10, and 11, in one possible embodiment, a first carrier plate 23 and a second carrier plate 24 may be provided on opposite sides of the first guide rail 221, respectively, and the carrying surfaces of the first carrier plate 23 and the second carrier plate 24, i.e., the surfaces that are in contact with the guide channel 63 may be backed away from the mounting panel 21, and the extension directions of the first carrier plate 23 and the second carrier plate 24 may be in the same direction as the first direction. As an example, the first rail 221 may have a first face 2211 and a second face 2212 facing opposite directions. The first face 2211 and the second face 2212 may be provided on opposite sides of the first rail 221, and the first carrier plate 23 and the second carrier plate 24 may be provided on the first face 2211 and the second face 2212, respectively. By setting the first carrier plate 23 and the second carrier plate 24 on opposite sides of the first guide rail 221, the guide channel 63 can move along the guide rail 22 while contacting the carrying surfaces of the first carrier plate 23 and the second carrier plate 24, so that the first carrier plate 23 and the second carrier plate 24 can provide support for the guide channel 63, which is conducive to the nutritional infusion tubing assembly 5 being assembled to the nutritional infusion device 1 along the guide rail 22 in a smooth manner.

As shown in FIGS. 7, 9, 10, and 11, in one possible embodiment, the first guide rail 221 may be provided with a first blocking plate 25 and a second blocking plate 26 on opposite sides of the end of the first guide rail 221 away from the second guide rail 222, and the blocking surfaces of both the first blocking plate 25 and the second blocking plate 26 may be oriented toward the first fixing member 4. As an example, the first blocking plate 25 and the second blocking plate 26 may be provided on opposite sides of the first end 2213 along a direction parallel to the mounting panel 21. When the peristaltic tube 51 of the nutritional infusion tubing assembly 5 is looped around the roller 3, the guide channel 63 of the nutritional infusion tubing assembly 5 can achieve initial alignment with the guide rail 221 through support from the first blocking plate 25 and second blocking plate 26. This prevents interference from the elastic recovery force of the nutritional infusion tubing assembly 5 during the initial engagement between the guide channel 63 and guide rail 22, thereby facilitating subsequent proper assembly of the nutritional infusion tubing 5 along the guide rails onto the nutritional infusion device 1.

As shown in FIGS. 7, 10 and 14, in one possible embodiment, the nutritional infusion device 1 may further include a fastening member 27 and a limiting member 28. The fastening member 27 may be disposed between the guide rail 22 and the roller 3, the fastening member 27 may abut against both the surface of the first blocking plate 25 and the surface of the second blocking plate 26 that face away from the first fixing member 4. The fastening member 27 may be provided with a tube groove 271, and the limiting member 28 may partially cover an opening of the tube groove 271. As an example, the opening may include a first opening 2711 along the length of the tube groove 271, and two second openings 2712 located at the upper and lower ends of the tube groove 271, so that the limiting member 28 may partially cover the first opening 2711 or partially cover the second openings 2712.

By providing the fastening member 27 and the limiting member 28 in the nutritional infusion device 1, in which the fastening member 27 may be disposed between the guide rail 22 and the roller 3 and may have a tube groove 271, after the nutritional infusion tubing assembly 5 is installed to the nutritional infusion device 1, the nutritional infusion tubing 52 (comprising input and output tubes) may be partially nested in the tube groove 271, which promotes the stability of the installation of the nutritional infusion tubing assembly 5. By causing the fastening member 27 to abut both the surfaces of the first blocking member 25 and the second blocking member 26 that face away from the first fixing member 4, any gap between the fastener 27 and the first blocking member 25 or the second blocking member 26 can be eliminated. This configuration improves the structural compactness of the nutritional infusion device 1 and prevents the need for cleaning residual nutrient droplets that may enter the gap. Furthermore, by arranging the limiting member 28 to partially cover the opening of the tube groove 271, the limiting member 28 can prevent the nutritional infusion tubing 52 nested within the tube groove 271 from dislodging after assembly. This ensures stable engagement between the nutritional infusion tubing assembly 5 and the nutritional infusion device 1.

As an example, the groove wall of the tube groove 271 may be provided with an ultrasonic bubble sensor 251. When the peristaltic tube 51 is slotted in the tube groove 271, the ultrasonic bubble sensor 251 is capable of transmitting and receiving ultrasonic waves, and then determining whether there are air bubbles present according to the difference between their reflection and propagation, so as to realize the detection of air bubbles in the peristaltic tube 51; this promotes the safety and reliability of the nutritional infusion system 10.

As an example, other detection devices such as pressure sensors 252 may also be provided within the nutritional infusion device 1 for detecting other parameters such as the obstruction pressure in the peristaltic tube 51 to realize the monitoring of the normal and smooth work of the nutritional infusion system 10, so as to make it easier for the operator to timely understand and control the working state of the nutritional infusion system 10; the invention is not limited to use of any particular detection device. In an embodiment of this invention, the two tube grooves 271 may be provided with ultrasonic bubble sensors 251 and pressure sensors 252, respectively, and FIG. 5 only schematically expresses the positions of the ultrasonic bubble sensors 251 and the pressure sensors 252, which may be designed according to the actual needs. As an example, the tube grooves 271 may be disposed on the top side of the roller 3 toward the nutritional infusion device 1, and the spacing between the two tube grooves 271 close to the end portions of the roller 3 may be greater than the spacing between the two tube grooves 271 away from the end portions of the roller 3, and the two tube grooves 271 close the end portions of the roller 3 may have a similar, flared opening structure. By configuring the shape of the tube grooves 271 and utilizing their effective forming effect on the nutritional infusion tubing assembly 5, the tube grooves 271 can help maintain the nutritional infusion tubing assembly 5 in a “teardrop-shaped” bending state. This facilitates smoother winding of the peristaltic tube 51 around the roller 3. In other embodiments, the relative position of the tube grooves 271 and the roller 3 can be flexibly set according to the actual needs, and the tube grooves 271 may also be in a straight line, curved shape or other shapes. In some other embodiments, the nutritional infusion device 1 may be provided without the tube groove 271.

As shown in FIGS. 8, 9, and 11, in one possible embodiment, the guide rail 22 may further include a third guide rail 223, which may connect the first guide rail 221 and the second guide rail 222, and an the third guide rail 223 may be directed to extend toward the mounting panel 21. By providing the third guide rail 223 in the guide rail 22 to connect the first guide rail 221 and the second guide rail 222, the guide channel 63 of the nutritional infusion tubing assembly 5 can be smoothly transitioned from the first guide rail 221 to the second guide rail 222 through the third guide rail 223. By orienting the third guide rail 223 to extend toward the mounting panel 21, after the nutritional infusion tubing assembly 5 is assembled to the nutritional infusion device 1 along the second guide rail 222, the third guide rail 223 can provide a certain support for the support plate 15 with the guide channel 63, and facilitate the nutritional infusion device 1 to be assembled to the nutritional infusion device 1.

As shown in FIGS. 8, 9, and 11, in a possible embodiment, the mounting panel 21 may include the mounting surface 212 and the recess 211. The mounting surface 212 may be configured to provide a carrying space for the support plate 15 of the nutritional infusion tubing assembly 5 to enter the guide rail 22, and the recess 211 may be configured to provide a carrying space for the support plate 15 of the nutritional infusion tubing assembly 5 to enter the guide rail 22 after the support plate 15 of the nutritional infusion tubing assembly 5 is fixed to the first fixing member 4. The guide rail 22 may be provided in the middle of the mounting surface 212 and the recess 211, separating the mounting surface 212 into a first mounting surface 2121 and a second mounting surface 2122, and separating the recess 211 into a first recess 2111 and a second recess 2112, which is more conducive to maintaining a balance during movement of the nutritional infusion tubing assembly 5. It should be noted that the guide rail 22 may not only be provided in the middle of the mounting surface 212 and the recess 211, i.e., the guide rail 22 separates the mounting panel 21 into two portions of equal area. Alternatively, the guide rail 22 may also be provided at ¼ of the mounting panel 21, i.e., the mounting panel 21 may be separated into two portions having an area ratio of 1:3. The guide rail 22 may be disposed at any position on the mounting panel 21 according to actual requirements, provided that it enables balanced and smooth assembly of both left and right sides of the nutritional infusion tubing assembly 5 onto the nutritional infusion device 1. By providing a mounting surface 212 on the mounting panel 21, during initial engagement between the guide channel 63 of the nutritional infusion tubing assembly 5 and the guide rail 22 of the nutritional infusion device 1, the guide channel 63 can partially contact the mounting surface 212 to obtain supporting force therefrom, thereby facilitating the initial engagement process. By providing the recess 211 in the mounting panel 21, after the nutritional infusion tubing assembly 5 is fully assembled on the nutritional infusion device 1, at least a partial wall of the recess can provide support to the support plate 15 with the guide channel 63; this improves the structural stability of the mating interface between the nutritional infusion tubing assembly 5 and the nutritional infusion device 1. By positioning the guide rail 22 intermediately between the mounting surface 212 and recess 211, the elastic resistance acting on the nutritional infusion tubing assembly 5 becomes symmetrically distributed. This configuration reduces the thrust required for moving the nutritional infusion tubing assembly 5 along the guide rail 22, consequently facilitating the smooth assembly of the nutritional infusion tubing assembly 5 on the nutritional infusion device 1 along the guide rail 22.

As shown in FIG. 7, in one possible embodiment, the mounting panel 21 may be integrally molded with the guide rail 22. By making the guide rail 22 integrally molded with the mounting panel 21, any gap between the guide rail 22 and the mounting panel 21 can be eliminated to avoid contamination caused by nutrient solution flowing into the gap, thereby reducing cleaning difficulty of the nutritional infusion device 1.

The structure of the nutritional infusion device 1 is described in detail above, and the detailed structure of the nutritional infusion tubing assembly 5 is described below.

As shown in FIGS. 7, 10, and 11, the nutritional infusion tubing assembly 5 may include the peristaltic tube 51, the infusion tube 52, the input tube connector 53, the output tube connector 54, the support plate 15, and the second fixing member 7. The infusion tube 52 may include the input tube 521 and the output tube 522. The input tube connector 53 may be connected to the input tube 521 and one end of the peristaltic tube 51, and the output tube connector 54 may be connected to the output tube 522 and the opposite end of the peristaltic tube 51. The support plate 15 may be provided with a first mounting groove 61, a second mounting groove 62, and the guide channel 63. The first mounting groove 61 may be configured to accommodate the input tube connector 53; the second mounting groove 62 may be configured to accommodate the output tube connector 54; and the guide channel 63 may be provided between the first mounting groove 61 and the second mounting groove 62 for cooperating with the guide rail 22 of the nutritional infusion device 1 so as to provide a guiding effect during movement of the support plate 15. The second fixing member 7 may be fixedly connected to a side of the support plate 15 back away from the peristaltic tube 51.

The nutritional infusion tubing assembly 5 is configured with the peristaltic tube 51, the inlet tube 521, the outlet tube 522, the inlet connector 53, the outlet connector 54, the support plate 15, and the second fixing member 7. The support plate 15 is provided with the first mounting groove 61 and the second mounting groove 62, which allows the inlet connector 53 that connects the inlet tube 521 to the peristaltic tube 51 to be accommodated in the first mounting groove 61, and the outlet connector 54 which connects the outlet tube 522 to the peristaltic tube 51 to be accommodated in the second mounting groove 62. This configuration enables stable positioning of the infusion tube 52 (including the inlet tube 521 and outlet tube 522) on the support plate 15. Furthermore, by providing the guide channel 63 on the support plate 15, after the peristaltic tube 51 is looped around the roller 3 of the nutrient infusion device 1, the restoring force acting on the nutritional infusion tubing assembly 5 is constrained to act primarily along the extension direction of the guide rail 22 through the cooperative interaction between the guide channel 63 and guide rail 22. In other words, the lateral components of the restoring force are effectively counteracted by the cooperative interaction between the guide channel 63 and guide rail 22. Consequently, the nutritional infusion tubing assembly 5 can be smoothly installed onto the nutritional infusion device 1 by applying only an axial force along the extension direction of the guide rail 22.

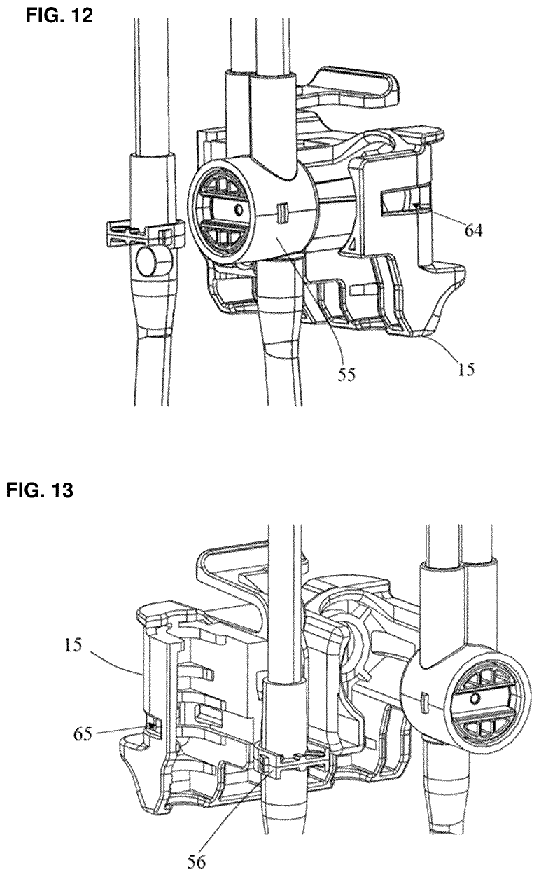

FIG. 12 is an exploded view of the nutritional infusion tubing assembly of FIG. 10, and FIG. 13 is another exploded view of the nutritional infusion tubing assembly of FIG. 10. As shown in FIGS. 11, 12, and 13, in one possible embodiment, the nutritional infusion tubing assembly 5 may further include a first mounting member 55 and a second mounting member 56. The first mounting member 55 may be fixedly connected to the input tube connector 53, and an inner wall of the first mounting groove 61 may be provided with a first locking member 64, so that the first mounting member 55 may form a detachable connection with the first locking member 64. The second mounting member 56 may be fixedly connected to the output tube connector 54, and an inner wall of the second mounting groove 62 may be provided with a second locking member 65, so that the second mounting member 56 may form a detachable connection with the second locking member 65. As an example, the first locking member 64 and the second locking member 65 may both have locking surfaces, and after the first mounting member 55 and the second mounting member 56 enter the first mounting groove 61 and the second mounting groove 62, respectively, the locking surfaces may form a locking relationship with the first mounting member 55 and the second mounting member 56. This configuration may enhance the strength of the connection of the input tube connector 53 to the first mounting groove 61, and the strength of the connection of the output tube connector 54 connected to the second mounting groove 62, so as to avoid the input tube 521 and the output tube 522 falling out of the first mounting groove 61 and the second mounting groove 62, respectively, which may ultimately help stabilize the mating structure of the infusion tube 52 and the support plate 15.

The nutritional infusion tubing assembly 5 having a three-groove structure with the first mounting groove 61, the second mounting groove 62, and the guide channel 63 is described above, and the mating structure of the nutritional infusion tubing assembly 5 formed after it is assembled in the nutrient infusion device 1 is described as below.

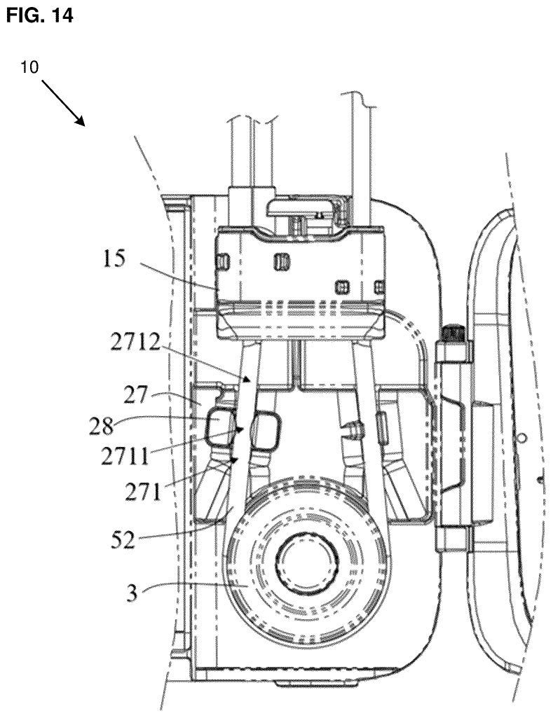

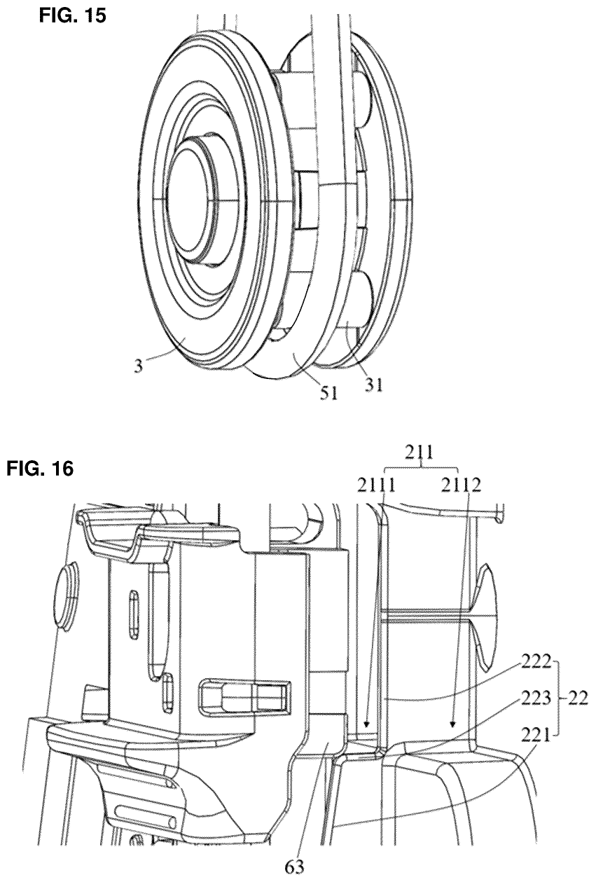

FIG. 14 is a schematic structural diagram of the nutritional infusion system according to some embodiments of this invention, FIG. 15 is a schematic diagram of the mating structure of the roller 3 and the peristaltic tube 51 according to some embodiments of this invention, and FIG. 16 is a schematic diagram of the mating structure of the guide rail 22 and the guide channel 63 according to some embodiments of this invention. As shown in FIGS. 14, 15, and 16, the nutritional infusion system 10 may include the nutritional infusion device 1 and the nutritional infusion tubing assembly 5, and the mounting panel 21 may be oriented toward the support plate 15. The peristaltic tube 51 may be looped around the rotors 31 of the roller 3, the guide rail 22 may be abutted against the inner wall of the guide groove 63, and the first fixing member 4 may be formed a detachable connection with the second fixing member 7.

The nutritional infusion system 10 comprises the nutritional infusion device 1 and the nutritional infusion tubing assembly 5 proposed in this invention, in which the mounting panel 21 may be oriented toward the support plate 15, the peristaltic tube 51 may be looped around the rotors 31 of the roller 3, and the guide rail 22 may abut the inner wall of the guide groove 63. During the process of stretching the nutritional infusion tubing assembly 5 for further assembly within the nutritional infusion device 1, the cooperative action between the guide channel 63 and the guide rail 22 can reduce the interference of elastic resistance to the assembly process, so that the nutritional infusion tubing assembly 5 only needs to be subjected to a force along the extension direction of the guide rail 22 to smoothly reach the final assembly position on the nutritional infusion device 1. Subsequently, through the detachable connection between the first fixing member 4 and the second fixing member 7, illustratively, after the nutritional infusion tubing assembly 5 completes sliding along the guide rail 22, the snap tab 71 of the second fixing member 7 can enter the snap groove 41 of the first fixing member 4, thereby forming a snap-fit connection with the snap groove 41. In addition, at least part of the wall of the recess 211 of the nutrient infusion device 1 can provide a supporting effect for at least a portion of the guide channel 63, which can realize a reliable cooperative working structure between the nutritional infusion tubing assembly 5 and the nutritional infusion device 1, namely the nutritional infusion system 10, ensuring the normal operation of the nutritional infusion system 10.

In the prior art, the process of assembling the nutritional infusion tubing assembly 5 on the nutritional infusion device 1 usually requires the operator to work with both hands to first overcome the elasticity of the peristaltic tube 51 and pull the peristaltic tube 51 in order to tightly loop it around the roller 3. Further, it is also necessary to rely on manually pressing the peristaltic tube 51 into the tube grooves 271 at a certain depth in order to make it possible to detect air bubbles in the peristaltic tube 51 and measure the obstruction pressure. Finally, the pump door 6 is closed, relevant parameters are set, and the nutritional infusion system 10 is started so as to feed the patient. The assembly process involves cumbersome steps and requires two-handed coordination by the operator, resulting in operational inconvenience.

Thus, embodiments of this invention provide a method of assembling the nutritional infusion tubing assembly 5 for the nutritional infusion device 1, which can simplify the steps of installing the nutritional infusion tubing assembly 5 in the nutritional infusion device 1. In addition, as shown in FIG. 2, the embodiments of this invention also provide a nutritional infusion system 10 with some structural improvements compared to the existing nutritional infusion system 10, which can also be beneficial for simplifying the steps of mounting the nutritional infusion tubing assembly 5 to the nutritional infusion device 1, and the nutritional infusion system 10 can be assembled by the assembly method provided by the embodiments of this invention. The assembly method provided in the embodiments of this invention may also be used for assembling the nutritional infusion system 10 of other realized structures, and the nutritional infusion system 10 provided in the embodiments of this invention may also be assembled by other assembly methods, which is not limited in this invention.

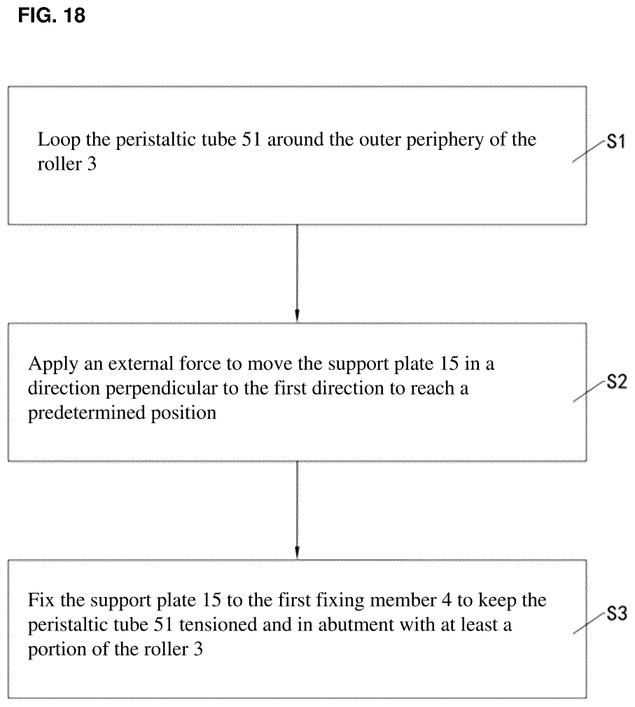

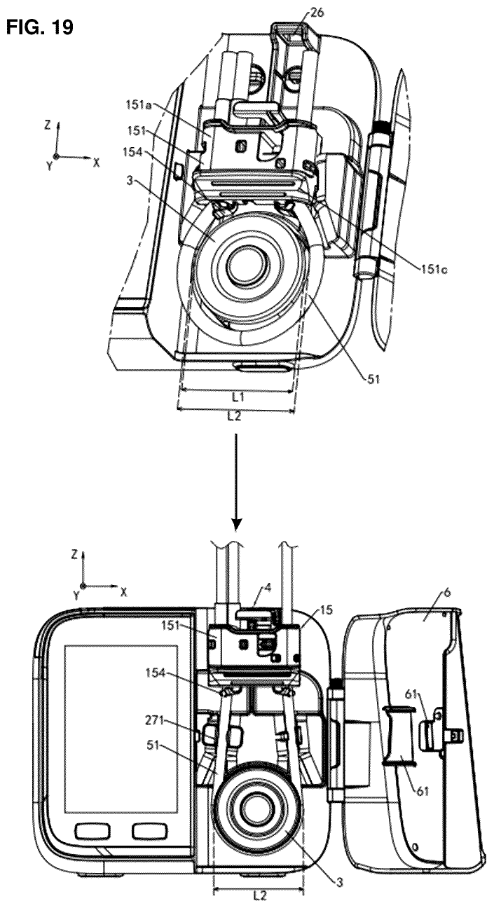

See FIGS. 18 and 19, of which FIG. 18 is a flowchart of a method of assembling the nutritional infusion system, and FIG. 19 is a schematic diagram of an assembly process of the nutritional infusion system made following the method shown in FIG. 18. The assembly method of this invention may generally include the following steps.

S1, loop the peristaltic tube 51 around an outer periphery of the roller.

The maximum spacing L1 of the peristaltic tube 51 in the first direction should be greater than or equal to the maximum spacing L2 of any two points on the roller 3. It should be understood that the maximum spacing L1 of the peristaltic tube 51 in the first direction refers to the maximum spacing L1 of the nutritional infusion tubing assembly 5 (as shown in FIG. 4) before the peristaltic tube 51 has been looped around the roller 3 (i.e., the peristaltic tube 51 is in a normal state or before it is deformed by an external force). By setting the maximum spacing L1 of the peristaltic tubes 51 in the normal state to be greater than or equal to the maximum spacing L2 between any two points on the roller 3 (as shown in FIG. 2), the peristaltic tubes 51 can be easily looped around the roller 3, which simplifies the process compared with the prior art, in which the user needs to overcome the elastic force of the peristaltic tubes 51 to hold them apart before they can be looped around the roller 3, and makes it possible for the user to loop the peristaltic tube 51 around the roller 3 with one hand, thereby simplifying the process of mounting the nutritional infusion tubing assembly 5 on the nutritional infusion device 1, and facilitating the operator's use.

The first direction may be parallel to the mounting surface 203 of the housing 21, for example, the first direction may be parallel to the X-axis direction. In other embodiments, the first direction may also be parallel to the Y-axis direction or the Z-axis direction or, the first direction may intersect the X-axis direction and/or the Y-axis direction and/or the Z-axis direction. In an embodiment of this invention, the horizontal direction of the input tube connector 12 and the output tube connector 13 may be parallel to the X-axis direction, i.e., the first direction may be parallel to the horizontal direction of the input tube connector 12 and the output tube connector 13. In other embodiments, the horizontal direction of the input tube connector 12 and the output tube connector 13 may intersect with the X-axis direction. In other embodiments, the input tube connector 12 and the output tube connector 13 may also be arranged in a direction intersecting the X-axis direction.

In an embodiment of this invention, the roller 3 may be approximately cylindrical, and the maximum spacing L2 of any two points on the roller 3 may also be understood to be the length of the diameter of the roller 3, i.e., the maximum spacing L1 of the peristaltic tube 51 in the first direction may be greater than or equal to the diameter of the roller 3 in the first direction (the maximum spacing L2), which can be realized by single-handedly looping the peristaltic tube 51 in the fixed state around the periphery of the roller 3. In this circumstance, the plane in which the peristaltic tube 51 lies may be considered to be parallel to the end face of the roller 3; the peristaltic tube 51 corresponds to the roller 3; and the lines of the two points with the maximum spacing on the roller 3 may be parallel to the first direction (i.e., the lines of the two points with the maximum spacing on the roller 3 may be parallel to the X-axis direction). A normal, “negligible deformation state” is the state of the peristaltic tube 51 when the operator has not applied additional external force to the peristaltic tube 51 and there is at most a slight deformation due to the support of other structures within the nutritional infusion tubing assembly 5.

In an embodiment of this invention, the diameter of the roller may be 33 mm. As an example, the size L1 of the peristaltic tube 51 in the first direction in the normal, unstressed state may be greater than or equal to 33 mm. In other embodiments, the maximum spacing L2 between any two points of the roller may be other values such as 30 mm, 32 mm, 35 mm, 38 mm, or more. The dimension L1 of the peristaltic tube 51 along the first direction in the normal state may be configured as required.

It should be understood that there may also be an angle between the direction of the line of any two points on the roller 3 and the first direction. In an embodiment of this invention, there may be an angle between the line of the two points with the maximum spacing on the roller 3 and the X-axis direction. For example, the cross-section of the surface of the roller 3 may also be oval, square, triangular, and other shapes, and the maximum spacing of any two points on the roller 3 may be the length of the long axis of the roller 3 or the length of the diagonal of the roller 3. During the process of looping the peristaltic tube 51 around the outer periphery of the roller 3, the peristaltic tube 51 may also be partially contacted by a portion of the outer circumference of the roller 3 in the negligible deformation state, using the contact points between the roller 3 and the peristaltic tube 51 as force-applying points, then slightly deforming the peristaltic tube 51 by moving the support plate 15 before contacting another portion of the peristaltic tube 51 with another portion of the outer periphery of the roller 3. The force for moving the support plate 15 to slightly deform the peristaltic tube 51 is much less than the maximum force that can be provided by one hand of the operator. In this circumstance, moving the support plate 15 can be understood as rotating or translating the support plate 15 at an arbitrary angle in order to adjust the support plate 15 to a state squarely opposite to the mounting surface 203, so that one side of the support plate 15 in the Z-axis direction can be parallel to the X-axis direction, or the arrangement direction of both sides of the support plate 15 in the X-axis direction may be parallel to the X-axis direction.

As shown in FIG. 4 and FIG. 19, in some embodiments, the support plate 15 may further include a support member 154, which may be disposed on a side of the plate body 151 facing toward the roller 3. The support member 154 may abut the peristaltic tubes 51 to hinder the different portions of peristaltic tubes 51 from coming too close to each other, so that the maximum spacing L1 between any two point the peristaltic tubes 51 in the first direction (i.e., the X-axis direction) in the normal state may be greater than or equal to any two points of the roller 3; in other words, the tube 51 “loop” is, at its widest, just as wide or wider than the roller 3. For example, the number of the support members 154 may be two, and the two support members 154 may be abutted against the input end 111 of the peristaltic tube 51 and to the output end 112 of the peristaltic tube 51, respectively, thereby preventing the output end 112 of the peristaltic tube 51 from approaching the input end 111 of the peristaltic tube 51 in the first direction, so that the maximum spacing L1 of the peristaltic tube 51 in the first direction in the natural state may be greater than or equal to the maximum spacing L2 between any two points on the roller 3. The input end 111 of the peristaltic tube 51 is an area or segment having a certain length, which may include a portion of the peristaltic tube 51 connected to the input tube connector 53, and may also include a tube of the peristaltic tube 51 in the vicinity of the input tube connector 53. Similarly, the output end 112 of the peristaltic tube 51 is a region or segment having a certain length, or may include a portion of the peristaltic tube 51 connected to the output tube connector 54, or may also include a tube of the peristaltic tube 51 in the vicinity of the output tube connector 54.

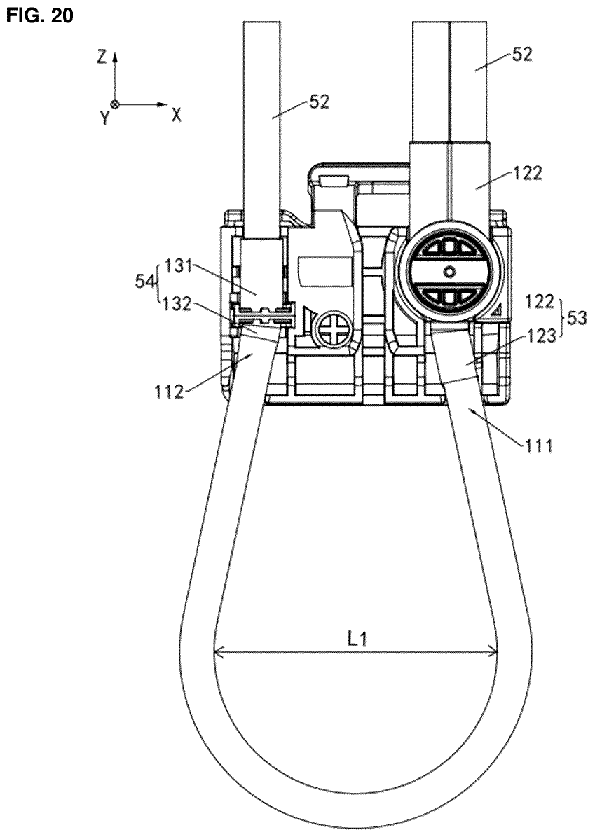

See FIG. 20, which is a schematic structural diagram of another nutritional infusion tube according to some embodiments of the present application.

In some embodiments, the nutritional infusion tubing assembly 5 may not include a support member 154, and the input tube connector 53 may include a first portion 122 and a second portion 123. The first portion 122 communicates with the infusion tube 52, and the second portion 123 communicates with an input end 111 of the peristaltic tube 51. The second portion 123 extends with respect to the first portion 122 toward a side away from the output tube connector 54; and/or, the output tube connector 54 may include a third portion 131 and a fourth portion 132, the fourth portion 132 being connected to the output end 112 of the peristaltic tube 51. The fourth portion 132 extends with respect to the third portion 131 towards the side away from the input tube connector 53. By changing the shape of the input tube connector 53 and/or the shape of the output tube connector 54, it is also possible to prevent the output end 112 of the peristaltic tube 51 from approaching the input end 111 in the first direction, thereby ensuring that the maximum spacing L1 of the peristaltic tube 51 along the first direction in the natural state is greater than or equal to the maximum spacing L2 between any two points on the roller 3.

In other embodiments, the peristaltic tube 51 may also be made to have a size L1 greater than the maximum spacing L2 between any two points of the roller 3 in the normal state by changing the shape of the snap groove or space, etc., on the support plate 15 in which the input tube connector 53 and/or the output tube connector 54 are mounted, which is not limited in this invention.

S2, apply an external force to move the support plate 15 along in a direction perpendicular to the first direction to reach a predetermined position.

In some embodiments, one of the nutritional infusion device 1 and the support plate 15 includes a guide rail, and the other includes a guide channel. In the step of applying the external force to move the support plate 15 away from the roller 3 to reach the predetermined position, the external force may be applied to move the support plate 15 in a direction perpendicular to the first direction to reach the predetermined position, following the engagement trajectory of the guide rail and guide channel. The cooperation between the guide rail and guide channel limits the movement path of the support plate 15, thereby improving the positional accuracy of the support plate 15 installation.

For example, as shown in FIGS. 4 and 5, in an embodiment of this invention, the nutritional infusion device 1 may include a guide rail 30, and the support plate 15 may include a guide channel 63. During the process of applying the external force to move the support plate 15 away from the roller 3, the guide rail 30 may be slotted in the guide channel 63, and the support plate 15 can move along the extension path of the guide rail 30. In other embodiments, the nutritional infusion device 1 may include a guide channel 63, and the support plate 15 may include the guide rail 30. During the process of applying the external force to move the support plate 15 away from the roller 3, the guide rail 30 may be slotted within the guide channel 63, and the support plate 15 may move along the extension path of the guide channel 63.

As an example, the guide rail 30 may include the first guide rail 301, the second guide rail 302, and the third guide rail 303. During the process of applying the external force along the guide rail 30 to move the support plate 15 along a direction perpendicular to the first direction to reach the predetermined position, the method may also include the following steps.

S21, apply the external force to move the support plate 15 along the first guide rail 301 in a direction away from the roller 3. The process is complete when the support plate 15 is detached from the first guide rail 301. In this circumstance, the support plate 15 moves along the second direction (i.e., along the direction of the Z-axis) with respect to the roller 3, and the support plate 15 drives the peristaltic tube 11 to move, causing the peristaltic tubes 11 around the roller 3 to gradually be stretched and elongated.

S22, apply an external force to move the support plate 15 along the third guide rail 303. The process is complete when the second guide rail 302 is able to extend into the guide channel 63 of the support plate 15. In the embodiment of this invention, after applying an external force to make the support plate 15 move along the third guide rail 303, the support plate 15 can be considered to have arrived at the predetermined position. During this process, the support plate 15 is able to drive the peristaltic tube 51 toward the mounting surface 203, which helps reduce the risk of the peristaltic tube 51 “flying out” from the periphery of the roller 3 and disengaging from the roller 3.

In other embodiments, after applying the external force to move the support plate 15 along the third rail 303, the method may also include: applying an external force to move support plate 15 along the second rail 302 for a certain distance to reach the predetermined position.

In some embodiments, as shown in FIG. 19, the support plate 15 may have a top surface 151a and a bottom surface 151b (as shown in FIG. 4), with the top surface 151a of the support plate 15 and the bottom surface 151b of the support plate 15 being located on two sides of the support plate 15 opposite to each other in the Y-axis direction, and the bottom surface 151b of the support plate 15 being closer to the mounting surface 203 relative to the top surface 151a of the support plate 15. The top surface 151a of the support plate 15 is provided with a sloped portion 151c, and the angle between the sloped portion 151c and the bottom surface 151b of the support plate 15 is an acute angle. In this embodiment of the invention, the sloped portion 151c may also be considered to extend upwardly from the side of the support plate 15 close to the roller 3 toward the side of the support plate 15 back from the roller 3.

The step of applying the external force to move the support plate 15 along a direction away from the roller 3 to reach the predetermined position may include: using the sloped portion 151c as a force-applying point, applying an external force move the support plate 15 along a direction away from the roller 3 to reach the predetermined position. By setting the slope 151c and using it as the force-applying point, the force applied by the operator to the sloped portion 151c can be decomposed into a force parallel to the mounting surface 203 of the nutritional infusion device 1 for pushing/pulling the support plate 15 to elongate the peristaltic tube 51, and a force perpendicular to the nutritional infusion device 1 to keep the support plate 15 close to the nutritional infusion device 1, so that the support plate 15 is always attached to the mounting surface 203 of the nutritional infusion device 1, thereby avoiding issues in the prior art where operators needed to manually hold both ends of the support plate 15 during movement, and where directional force components could easily cause the support plate 15 to detach from the nutritional infusion device 1. This significantly improves the reliability of the installation process.

S3, fix the support plate 15 to the first fixing member 4 to keep the peristaltic tube 51 tensioned and in abutment with at least a portion of the roller 3. The tensioned state of the peristaltic tube 51 refers to the condition where the tube is straightened but before reaching the breaking point when further stretched.

When fixing the support plate 15 to the first fixing member 4 to keep the peristaltic tube 51 tensioned and in abutment with at least a portion of the roller 3, two ends of the peristaltic tube 51 are symmetrically disposed with respect to the roller 3, i.e., in the embodiment of this invention, the direction of a symmetry axes of the two ends of the peristaltic tube 51 may be parallel to the Z-axis direction.

In an embodiment of this invention, the second fixing member 152 has a certain clastic deformation capacity. During the installation process, an external force is applied to squeeze the second fixing member 152 on the support plate 15 into the first fixing member 4 and snap the second fixing member 152 into engagement with the first fixing member 4, thereby fixing the second fixing member 7 of the support plate 15 to the first fixing member 4 to keep the peristaltic tube 51 tensioned and in abutment with at least a portion of the roller 3. In addition, during the disassembly process, the separation of the second fixing member 152 from the first fixing member 4 can also be easily achieved by pushing the second fixing member 152 in the reverse direction.

In an embodiment of this invention, the second fixing member 152 is provided on a side of the support plate 15 back away from the roller 3. In the aforementioned step S22, an external force is applied to squeeze the second fixing member 152 into the first fixing member 4 while the support plate 15 is moving along the third rail 303. When the support plate 15 is located in the predetermined position, the second fixing member 152 is just able to be slotted in the first fixing member 4, the locking member 17 is slotted in the locking hole 153, and the switching member 18 is slotted in the reversing groove 121. In other words, during the process of moving the support plate 15 to reach the predetermined position, the process of fixing the support plate 15 with the first fixing member 4 is also realized, which simplifies the operation steps, facilitates the use by the operator, and makes for a faster and simpler assembly process for the nutritional infusion system 10.

In other embodiments, after applying the external force to move the support plate 15 along the third guide rail 223, the second fixing member 7 may not be completely slotted in the first fixing member 4. The external force may then continue to be applied to move the support plate 15 along the second guide rail 222 for a certain distance, so that the second fixing member 7 is completely slotted in the first fixing member 4, and the support plate 15 reaches the predetermined position.

In some embodiments, the pump door 6 is provided with tube-pressing blocks 601, and the assembly method may also include the following steps.

S4, close the pump door 6 so that the tube pressing blocks 601 push the peristaltic tube 51 into the corresponding tube grooves 271. By providing the tube pressing blocks 601 on the pump door 6, the peristaltic tube 51 can be pressed into the tube grooves 271 while closing the pump door 6, which is convenient for the operator to use and eliminates the need for the operator to take an additional step of manually pressing the peristaltic tube 51 into the tube grooves 271 as in the prior art, and is helpful for realizing a faster and simpler assembly process of the nutritional infusion system 10. As an example, the number of tube-pressing blocks 601 may be two, and these may be spaced apart on the pump door 6, so that the two tube-pressing blocks 601 can separately press a portion of the peristaltic tubes 11 into the tube grooves 271 while the pump door 6 is closed.

In some embodiments, after the step S3 (the support plate 15 is fixed with the first fixing member 4 to keep the peristaltic tube 51 tensioned and in abutment with at least a portion of the roller 3), or after the step S4 (the pump door 6 is closed, and the presser block 221 pushes the peristaltic tube 51 into the corresponding tube groove 271, the assembly method may also include: manipulating the nutritional infusion device 1 by the button 292 to activate the second motor within the nutritional infusion device 1 connected to the locking member 17 to further fix the relative position of the support plate 15 and the nutritional infusion device 1, thereby improving the assembly reliability of the nutritional infusion system 1.

Described above are only some of the embodiments and implementations of this invention, and the scope of protection of this invention is not limited thereto. Any skilled person skilled in the art can easily think of changes or modifications within the scope of the technology disclosed in this invention, which shall be covered within the scope of protection of this invention. Therefore, the scope of protection of this invention shall be subject to the scope of protection of the claims.

1. A nutritional infusion device, comprising:
a housing;
a power supply and a control assembly provided inside the housing;
a driving assembly connected to the control assembly and provided inside the housing;
a roller connected to and rotatable by the driving assembly and exposed above a mounting panel of the housing; wherein the roller is configured
to receive a peristaltic tube looped around it;
under the driving force of the driving assembly, the roller is configured to drive a plurality of rotors set in the roller to sequentially squeeze the peristaltic tube so as to cause a directional movement of a liquid inside the peristaltic tube; and
a first fixing member;
wherein the mounting panel is provided with a guide rail assembly that is configured to guide the movement of a nutritional infusion tubing assembly with respect to the mounting panel after the peristaltic tube has been looped around the roller and before it is fixed to the first fixing member.

2. The nutritional infusion device of claim 1, wherein the mounting panel is provided with a recess, and at least a partial wall of the recess is configured to abut against at least a partial portion of a support plate of the nutritional infusion tubing assembly after the support plate of the nutritional infusion tubing assembly has been fixed to the first fixing member.

3. The nutritional infusion device of claim 1, wherein the guide rail assembly comprises a first guide rail and a second guide rail, wherein the first guide rail is disposed closer to the roller with respect to the second guide rail, and a direction of the roller towards the first fixing member is defined as a first direction;
wherein the first guide rail and the second guide rail extend parallel to each other.

4. The nutritional infusion device of claim 3, wherein the first guide rail and the second guide rail are provided in a stepped manner on the mounting panel.

5. The nutritional infusion device of claim 3, wherein
a first carrier plate and a second carrier plate are provided on opposite sides of the first guide rail;
a carrying surface of the first carrier plate and a carrying surface of the second carrier plate are offset inward, away from the mounting panel, and an extension direction of the first carrier plate and an extension direction of the second carrier plate are in the same direction as the first direction.

6. The nutritional infusion device of claim 3, wherein a first blocking plate and a second blocking plate are provided on opposite sides of an end of the first guide rail away from the second guide rail, and a blocking surface of the first blocking plate and a blocking surface of the second blocking plate are both facing the first fixing member.

7. The nutritional infusion device of claim 3, wherein
the guide rail assembly further comprises a third guide rail that is configured to connect the first guide rail and the second guide rail, and
the third guide rail extends in a direction towards the mounting panel.

8. The nutritional infusion device of claim 6, wherein the nutritional infusion device further comprises
a fastening member and a limiting member, wherein
the fastening member is disposed between the guide rail and the roller, and
the fastening member abuts a surface of the first blocking plate behind and separated from the first fixing member and a surface of the second blocking plate being located behind and separated from the first fixing member;
the fastening member is provided with a tube groove, and
the limiting member is configured to partially cover an opening of the tube groove.

9. The nutritional infusion device of claim 8, further comprising a pump door, wherein the pump door is provided with a tube-pressing block, and two tube grooves are provided between the roller and the fixing assembly.

10. The nutritional infusion device of claim 1, further comprising a housing, wherein
the fixing assembly is fixed to the housing, and the roller is rotatably connected to the housing;
the housing is provided with a mounting surface, and the support plate is mounted to the mounting surface;
the support plate is provided with a top surface and a bottom surface, which are disposed on opposing sides of the support plate;
the bottom surface is disposed closer to the mounting surface relative to the top surface, and the top surface is provided with a sloped portion, which forms an acute angle with the bottom surface.

11. The nutritional infusion device of claim 1, wherein
the mounting panel comprises a mounting surface and a recess, wherein the mounting surface is configured to provide a carrying space for the support plate of the nutritional infusion tubing assembly to enter the guide rail, and the recess is configured to provide a carrying space for the support plate of the nutritional infusion tubing assembly after it has been fixed to the first fastening member;
the guide rail is provided in the middle of the mounting surface and in the middle of the recess, dividing the mounting surface into a first mounting surface and a second mounting surface, and dividing the recess into a first recess and a second recess.

12. The nutritional infusion device of claim 1, wherein the mounting surface is integrally molded with the guide rail.

13. A nutritional infusion tubing assembly, comprising:
a peristaltic tube;
infusion tubing comprising an input tube and an output tube;
an input tube connector connected to the input tube and one end of the peristaltic tube;
an output tube connector connected to the output tube and another end of the peristaltic tube;
a support plate provided with a first mounting groove and a second mounting groove, wherein the first mounting groove is configured to accommodate the input tube connector, and the second mounting groove is configured to accommodate the output tube connector; and
a second mounting member fixedly connected to a side of the support plate behind and separated from the peristaltic tube.

14. The nutritional infusion tubing assembly of claim 13, wherein the support plate is provided with a guide channel, which is provided between the first mounting groove and the second mounting groove for cooperating with a guide rail of a nutritional infusion device for providing a guiding effect during movement of the support plate.

15. The nutritional infusion tubing assembly of claim 13, further comprising
a first mounting member and a second mounting member;
wherein
the first mounting member is fixedly connected to the input tube connector,
a first locking member is provided on an inner wall of the first mounting groove and is detachably connected to the first locking member;
the second mounting member is fixedly connected to the output tube connector;
a second locking member is provided on the inner wall of the second mounting groove; and
the second mounting member is detachably connected to the second locking member.

16. A nutritional infusion system, comprising:
a nutritional infusion device comprising:
a housing;
a power supply and a control assembly provided inside the housing;
a driving assembly connected to the control assembly and provided inside the housing;
a nutritional infusion tubing assembly including a peristaltic tube;
a roller connected to the driving assembly and exposed above a mounting panel of the housing; wherein the roller is configured to receive a peristaltic tube of the nutritional infusion tubing assembly to be looped around it; under the driving force of the driving assembly, the roller is configured to drive a plurality of rotors set in the roller to sequentially squeeze the peristaltic tube so as to cause a directional movement of a liquid inside the peristaltic tube; and
a first fixing member;
wherein the mounting panel is provided with a guide rail, which is configured to guide the movement of the nutritional infusion tubing assembly with respect to the mounting panel after the peristaltic tube has been looped, around the roller and before it is fixed to the first fixing member, wherein a maximum spacing between any two points of the peristaltic tube in a first direction is greater than or equal to a maximum spacing of any two points on the roller; and
the nutritional infusion tubing assembly further comprising:
an input tube and an output tube;
an input tube connector connected to the input tube and one end of the peristaltic tube;
an output tube connector connected to the output tube and another end of the peristaltic tube;
a support plate provided with a first mounting groove, a second mounting groove and a guide channel, wherein the first mounting groove is configured to accommodate the input tube connector, the second mounting groove is configured to accommodate the output tube connector, and the guide channel is provided between the first mounting groove and the second mounting groove for cooperating with the guide rail of the nutritional infusion device for providing the guiding effect during movement of the support plate; and
a second mounting member fixedly connected to a side of the support plate back away from the peristaltic tube;
wherein the mounting panel faces the support plate, the peristaltic tube is looped on the rotor of the roller, the guide rail abuts an inner wall of the guide channel, and the first fixing member is detachably connected to the second fixing member.

17. The nutritional infusion system of claim 16, wherein
the first mounting member comprises a snap groove, and the second mounting member comprises a snap tab;
during the movement of the support plate along the guide rail, while the support plate is displaced in a direction toward an interior of the housing, a tip of the snap tab is deformed by extrusion of the snap groove, and springs back upon reaching an opening of the snap groove, causing the snap groove and the snap tab to be fixed to each other.

18. The nutritional infusion system of claim 16, wherein the mounting panel is provided with a guide rail assembly, that is configured to guide the movement of the nutritional infusion tubing assembly with respect to the mounting panel after the peristaltic tube has been looped around the roller and before it is fixed to the first fixing member.

19. The nutritional infusion system of claim 16, wherein, after the peristaltic tube is looped on the roller, the support plate is movable in a direction perpendicular to the first direction under external force until reaching a predetermined position.

20. The nutritional infusion system of 16, wherein the support plate further comprises a support member, which is disposed on a side of a plate body facing toward the roller, and abuts the peristaltic tube to hinder different portions of the peristaltic tube from approaching each other.
